(19) United States
(12) Patent Application Publication
(10) Pub. No.: US 2025/0205067 A1
(43) Pub. Date: Jun. 26, 2025

(54) ENDOVASCULAR DELIVERY SYSTEMS WITH SERIALLY LOADED ENDOANCHORS

(71) Applicant: Medtronic Vascular, Inc., Santa Rosa, CA (US)

(72) Inventors: Julie-Anne McBARRON, Galway (IE); Dmitry SHEREMETIEV, Galway (IE); Ciaran M. McGUINNESS, Galway (IE); Sumeeyae HARIBABU, Galway (IE); Igor HEINZE, Galway (IE); Jeremy KINKADE, Santa Rosa, CA (US); Mark STIGER, Santa Rosa, CA (US); Emily GRIMM, Santa Rosa, CA (US)

(73) Assignee: Medtronic Vascular, Inc., Santa Rosa, CA (US)

(21) Appl. No.: 18/974,026

(22) Filed: Dec. 9, 2024

Related U.S. Application Data
(60) Provisional application No. 63/613,494, filed on Dec. 21, 2023.

(51) Int. Cl. A61F 2/95

(52) U.S. Cl. CPC A61F 2/9517; A61F 2220/0016

(57) ABSTRACT

An endoanchor delivery system comprising a handle system, an applier catheter, and a series of endoanchors. The applier catheter extends from the handle system and defines a lumen therein. The series of endoanchors are loaded into the lumen of the applier catheter at the distal end thereof and are disposed along a longitudinal axis of the applier catheter in a loaded state. The series of endoanchors include a first endoanchor and a second endoanchor. The first endoanchor is located distalmost the applier catheter in the loaded state. The second endoanchor is located inward the first endoanchor along the longitudinal axis of the applier catheter. The first endoanchor has a straight, flat shape in the loaded state. The first endoanchor has a helical, flat shape in a deployed state in which the first endoanchor anchors an implant to a blood vessel wall.

### CROSS-REFERENCE TO RELATED APPLICATIONS

This application claims the benefit of U.S. Provisional Patent Application Ser. No. 63/613,494, filed Dec. 21, 2023, the entire content of which is incorporated herein by reference.

### TECHNICAL FIELD

The present disclosure relates to endovascular delivery systems with serially loaded endoanchors.

### BACKGROUND

An aortic aneurysm may be an enlarged area in an aorta (e.g., an abdominal aorta). The aortic aneurysm may form from the degradation of elastin and/or interstitial collagen, which may alter the structural integrity of the aortic wall, which may weaken it. One current treatment of an aortic aneurysm may be endovascular surgery which utilizes an implant (e.g., stent graft). Another more invasive option is open surgery.

Endovascular procedures are minimally invasive techniques to deliver clinical treatments in a patient's vasculature (e.g., treatment of aortic aneurysms). One example of a clinical treatment used in an endovascular procedure is deployment of a stent graft. A conventional stent graft typically includes a radially expandable reinforcement structure, e.g., formed from a plurality of annular stent rings, and a cylindrically shaped layer of graft material defining a lumen to which the stent rings are coupled. The stent graft is placed inside a patient's vasculature (e.g., blood vessel) to bridge a diseased blood vessel segment (e.g., an aneurismal, dissected, or torn blood vessel segment), and thereby excluding hemodynamic pressures of blood flow from the diseased blood vessel segment.

The proximal end of the stent graft is expended onto a landing zone of the diseased blood vessel. The anatomy of the landing zone may compromise the fixation and sealing of the stent graft, thereby potentially causing leaking and/or movement of the stent graft. Staples may be applied to the proximal end of the stent graft to anchor the stent graft to the blood vessel wall.

### SUMMARY

In an embodiment, an endoanchor delivery system comprising a handle system, an applier catheter, and a series of endoanchors is disclosed. The applier catheter extends from the handle system and defines a lumen therein. The applier catheter has a proximal end and a distal end. The series of endoanchors are loaded into the lumen of the applier catheter at the distal end thereof and are disposed along a longitudinal axis of the applier catheter in a loaded state. The series of endoanchors include a first endoanchor and a second endoanchor. The first endoanchor is located distalmost the applier catheter in the loaded state. The second endoanchor is located inward the first endoanchor along the longitudinal axis of the applier catheter. The first endoanchor has a straight, flat shape in the loaded state. The first endoanchor has a helical, flat shape in a deployed state in which the first endoanchor anchors an implant to a blood vessel wall.

The first endoanchor may include a first surface and a second surface opposing the first surface. The first surface and/or the second surface may have a flat shape characteristic. In one or more embodiments, the first endoanchor in the deployed state has a first endoanchor deployed circumference, and the applier catheter has an outer surface and an inner surface, and the inner surface has an inner surface circumference less than the first endoanchor deployed circumference. The helical, flat shape may be a pre-set helical, flat shape. The first endoanchor may be formed of a shape memory material to set the pre-set helical, flat shape. The straight, flat shape of the first endoanchor in the loaded state includes a straight shape characteristic where the first endoanchor substantially follows the longitudinal axis of the applier catheter with deviations of 5% or less. The helical, flat shape of the first endoanchor in the deployed state includes a helical shape characteristic of a circular helix shape having a constant radius, curvature, and/or torsion. The endoanchor delivery system may further include a plunger configured to apply force to the series of endoanchors along the longitudinal axis of the applier catheter to transition the first endoanchor from the loaded state to the deployed state.

In another embodiment, an endoanchor delivery system is disclosed. The endoanchor delivery system includes a handle system, an applier catheter, and a series of endoanchors. The applier catheter extends from the handle system and defines a lumen therein. The applier catheter includes an inner shaft having a proximal portion and a distal end. The series of endoanchors is loaded into the lumen of the applier catheter and onto the distal end of the inner shaft thereof in a loaded state. The series of endoanchors includes a first endoanchor and a second endoanchor. The first endoanchor is located distalmost the applier catheter in the loaded state. The second endoanchor is located inward the first endoanchor along a longitudinal axis of the applier catheter. The first endoanchor includes a base and first and second legs extending from the base. The base defines an opening configured to communicate with the inner shaft for axial movement along the longitudinal axis. The first and second legs are in a loaded position in the loaded state. The first and second legs are in a deployed position different than the loaded position in a deployed state in which the first endoanchor anchors an implant (e.g., a stent graft) to a blood vessel wall.

The first endoanchor may include one or more ramps configured to communicate with the inner shaft for axial movement along the longitudinal axis. The first endoanchor may include one or more angled tabs configured to pierce the implant when the first endoanchor is in the deployed state. The base of the first endoanchor may be configured to contact the implant when the first endoanchor is in the deployed state. The first and second legs may be outwardly curving relative to the base of the first endoanchor. The distal end of the inner shaft may include a helical gear and the proximal portion of the inner shaft may have a cylindrical shape and does not include the helical gear. In one or more embodiments, all the endoanchors in the series of endoanchors are situated on the helical gear of the distal end of the inner shaft in the loaded state. The endoanchor delivery system may include one or more fixtures extending from an inner surface of the applier catheter. The one or more fixtures is configured to resist a rotation of the first and second endoanchors when the helical gear of the inner shaft is rotated and to permit axial movement of the first and second endoanchors along the longitudinal axis of the applier catheter.

In yet another embodiment, a method of deploying a series of endoanchors is disclosed. The method includes the step of loading a series of endoanchors serially within a lumen defined by a distal end of an applier catheter. The series of endoanchors includes a first endoanchor and a second endoanchor. The first endoanchor is located distalmost the applier catheter in a loaded state. The second endoanchor is located inward the first endoanchor along a longitudinal axis of the applier catheter. The first endoanchor includes a constrained shape and a pre-set shape. The first endoanchor is in the constrained shape in the loaded state. The method further includes sequentially deploying the series of endoanchors including expelling the first endoanchor from the lumen of the applier catheter such that the first endoanchor transitions from the constrained shape into the pre-set shape in which the first endoanchor anchors an implant to a blood vessel wall.

In connection with the method, the first endoanchor may include a proximal end a distal end. The sequentially deploying step for the first endoanchor may include a first partially deploying step and a second partially deploying step. The first partially deploying step may include contacting the stent graft with the distal end of the first endoanchor. The second partially deploying step may include piercing the implant and the blood vessel wall with the first endoanchor. The first and second endoanchors may be spaced apart from each other in the constrained shape along the longitudinal axis of the applier catheter. The loading step includes loading the series of endoanchors from a distal opening of the applier catheter.

### BRIEF DESCRIPTION OF THE DRAWINGS

FIG. 1A is a perspective view of a prior art endoanchor delivery system including an endoanchor guide and an endoanchor applier.

FIG. 1B is a perspective view of a prior art endoanchor cassette defining apertures for receiving endoanchors.

FIG. 1C is a cross section view of an abdominal aorta and a side view of prior art endoanchor catheter in a deployment state deploying an endoanchor to anchor a stent graft to a wall of the abdominal aorta.

FIG. 1D is a magnified view of a distal portion of the endoanchor guide in a deployment state deploying the endoanchor shown in FIG. 1C.

FIG. 2A is a cross section view of an applier catheter of an endoanchor delivery system and a side view of endoanchors loaded into the endoanchor catheter in a loaded state.

FIG. 2B is a cross section view of the applier catheter of FIG. 2A with an endoanchor in a first partially deployed state.

FIG. 2C is a cross section view of the applier catheter of FIG. 2A with the endoanchor of FIG. 2B in a second partially deployed state.

FIG. 2D is a cross section view of the applier catheter of FIG. 2A with the endoanchor of FIG. 2B in a deployed state.

FIG. 3A are perspective, side views of an endoanchor transitioning from a loaded state into a deployed state.

FIG. 3B is a perspective view of an applier catheter including endoanchors loaded into the endoanchor catheter in a loaded state.

FIG. 3C is a perspective view of the applier catheter of FIG. 3B with an endoanchor in a first partially deployed state.

FIG. 3D is a perspective view of the applier catheter of FIG. 3B with the endoanchor of FIG. 3C in a second partially deployed state.

FIG. 3E is a perspective view of the applier catheter of FIG. 3B with the endoanchor of FIG. 3C in a deployed state.

FIG. 4A is a cross section view of an applier catheter and a side view of endoanchors loaded into the applier catheter in a loaded state.

FIG. 4B is a side view of an endoanchor of FIG. 4A in a deployed state within a stent graft and a blood vessel wall.

FIG. 5A is a perspective view of an endoanchor having a base and first and second legs extending from the base.

FIG. 5B is a perspective view of the endoanchor of FIG. 5A in a loaded state in an applier catheter and mounted on a distal end of an inner shaft of an endoanchor delivery system.

FIG. 5C is a cross section view of the endoanchor of FIG. 5A in a deployed state anchoring a stent graft to a wall of a blood vessel (e.g., an abdominal aorta).

FIG. 5D is a cross section view of an applier catheter including rails matching notches in an endoanchor.

FIG. 5E is a cross section view of the applier catheter taken along line 5E-5E of FIG. 5D and showing the rails matching notches of the endoanchor.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

Directional terms used herein are made with reference to the views and orientations shown in the exemplary figures. A central axis is shown in the figures and described below. Terms such as “outer” and “inner” are relative to the central axis. For example, an “outer” surface means that the surfaces faces away from the central axis, or is outboard of another “inner” surface. Terms such as “radial,” “diameter,” “circumference,” etc. also are relative to the central axis. The terms “front,” “rear,” “upper” and “lower” designate directions in the drawings to which reference is made.

Unless otherwise indicated, for the delivery system the terms “distal” and “proximal” are used in the following description with respect to a position or direction relative to a treating clinician. “Distal” and “distally” are positions distant from or in a direction away from the clinician, and “proximal” and “proximally” are positions near or in a direction toward the clinician. For the stent-graft prosthesis, “proximal” is the portion nearer the heart by way of blood flow path while “distal” is the portion of the stent-graft further from the heart by way of blood flow path.

The following detailed description is merely exemplary in nature and is not intended to limit the invention or the application and uses of the invention. Although the description is in the context of treatment of blood vessels such as the aorta, coronary, carotid, and renal arteries, the invention may also be used in any other body passageways (e.g., aortic valves, heart ventricles, and heart walls) where it is deemed useful.

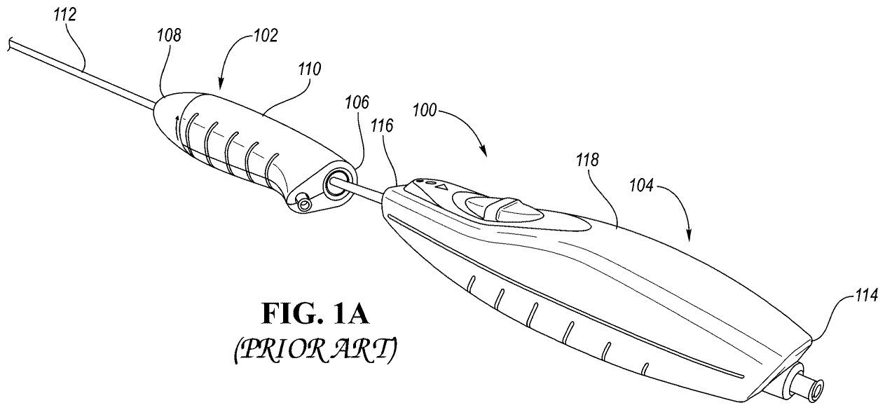

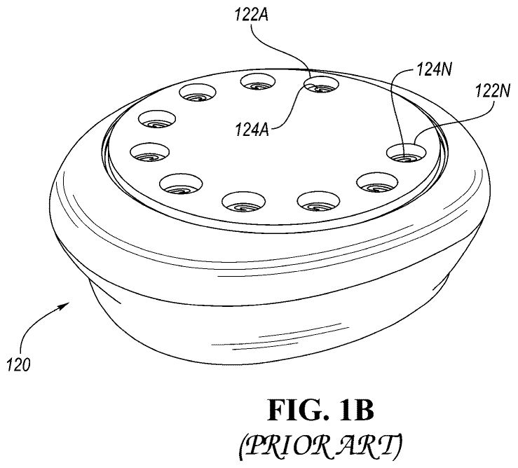

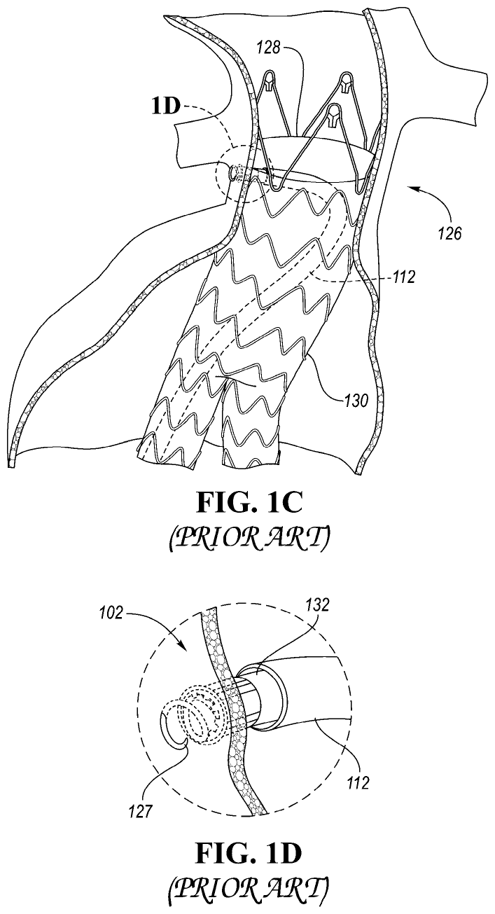

FIG. 1A is a perspective view of prior art endoanchor delivery system 100 including endoanchor guide 102 and endoanchor applier 104. Endoanchor guide 102 includes proximal end 106 and distal end 108 and body 110 extending therebetween. Endoanchor sheath 112 extends from distal end 108 of endoanchor guide 102. Endoanchor applier 104 include proximal end 114 and distal end 116 and body 118 extending therebetween.

FIG. 1B is a perspective view of prior art endoanchor cassette 120 defining apertures 122A through 122N for receiving endoanchors 124A through 124N, respectively. One of endoanchors 124A through 124N is loaded into a distal end of endoanchor guide 102. Endoanchor delivery system 100 is configured to deliver the loaded endoanchor to a delivery site to provide fixation and sealing between an endovascular implant (e.g., an endovascular stent graft) and a wall of native blood vessel (e.g., a thoracic aorta or an abdominal aorta). Endoanchor delivery system 100 is withdrawn from the vasculature of a patient after deployment of the loaded endoanchor so that a subsequent endoanchor may be loaded into the distal end of endoanchor guide 102.

FIG. 1C is a cross section view of abdominal aorta 126 and a side view of endoanchor sheath 112 in a deployment state deploying loaded prior art endoanchor 127 to anchor proximal end 128 of stent graft 130 to a wall of abdominal aorta 126. FIG. 1D is a magnified view of a distal portion of prior art endoanchor guide 102 in a deployment state deploying loaded endoanchor 127. The distal portion of endoanchor guide 102 includes endoanchor sheath 112 and endoanchor applier 132. As shown in FIG. 1C, endoanchor applier 132 is curved by deflectable endoanchor sheath 112 such that endoanchor applier 132 and endoanchor 127 are apposed against stent graft 130. Distal portion of endoanchor applier 132 is about 90 degrees relative to stent graft 130 such that endoanchor applier 132 reaches stent graft 130.

Challenging anatomy may make deployment of endoanchors difficult. The acute angulation in the abdominal aorta and/or the overall aorta size may make proper deflection and orientation of the staple application system difficult. Also, after one endoanchor is deployed, then the delivery system is removed from the patient's vasculature to insert another endoanchor, thereby creating successive, independent deployment sequences, making repeatability of proper deflection and orientation challenging.

In light of the foregoing, what is needed is an endoanchor delivery system in which multiple (e.g., two or more endoanchors) endoanchors may be loaded into the same applier catheter where the endoanchors may be deployed with proper positioning successively during one procedure (e.g., without removing the applier from the patient's anatomy to reload subsequent endoanchors).

In one or more embodiments, an endoanchor delivery system is disclosed. The endoanchor delivery system includes an endoanchor guide and an endoanchor applier. The endoanchor applier includes an applier catheter configured to load multiple (e.g., two or more endoanchors) endoanchors for deployment successively during one procedure. The endoanchor guide may be deflectable, such that the tip may have an angle of 90 degrees or less relative to the longitudinal axis (e.g., perpendicular or acute). This system and the related method have one or more benefits (e.g., reduced procedure time and/or increased accuracy of positioning of one or more of the multiple endoanchors).

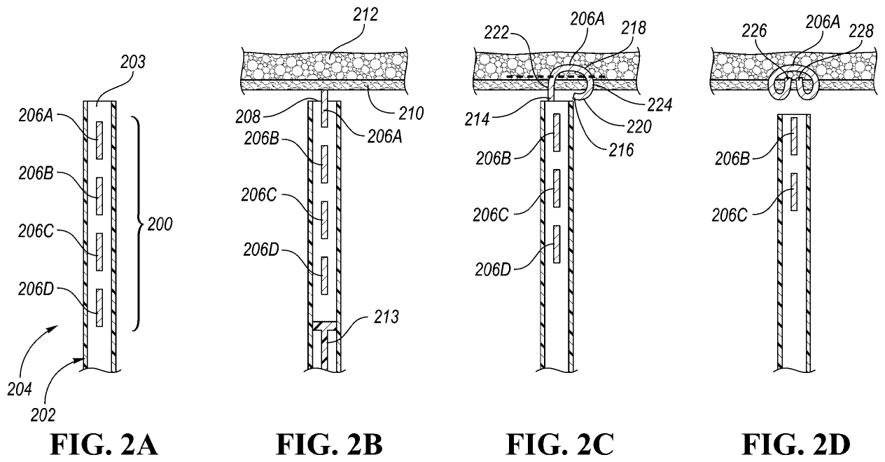

FIG. 2A is a cross section view of distal region 200 of applier catheter 202 of endoanchor delivery system 204. FIG. 2A depicts first endoanchor 206A, second endoanchor 206B, third endoanchor 206C, and fourth endoanchor 206D (e.g., staples) loaded into distal region 200 of applier catheter 202 in a loaded state. The endoanchors may have a pre-set shape outside of applier catheter 202. The endoanchors may be formed of a shape memory material or alloy (e.g., Nitinol). In one or more embodiments, upon loading into applier catheter 202, the endoanchors transition into a straight configuration (e.g., constrained configuration) to occupy the reduced volume defined by lumen 203. With the straight configuration, multiple endoanchors may be stacked within applier catheter 202 along the longitudinal axis of applier catheter 202. As shown in FIG. 2A, first endoanchor 206A, second endoanchor 206B, third endoanchor 206C, and fourth endoanchor 206D may be spaced apart from each other in the straight configuration. In other embodiments, one or more adjacent endoanchors may contact each other or partially overlap each other along the longitudinal axis of applier catheter 202. The straight, stacked configuration (e.g., as shown in FIG. 2A) permits the deployment of multiple endoanchors during a single procedure without removing and reloading applier catheter 202.

FIG. 2B depicts a side view of endoanchor 206A in a first partially deployed state. As shown in FIG. 2B, endoanchor 206A partially extends beyond distal end 208 of applier catheter 202. In the first partially deployed state, endoanchor 206A may contact stent graft 210, which is deployed against blood vessel wall 212. Plunger 213 may advance first endoanchor 206A (as well as endoanchor 206B, endoanchor 206C, and endoanchor 206D) through lumen 203 of applier catheter 202. Plunger 213 may push the proximal most endoanchor, which in turn, pushes forward the adjacent endoanchor, where the force is eventually transferred to endoanchor 206A.

FIG. 2C depicts a side view of endoanchor 206A in a second partially deployed state. As shown in FIG. 2C, endoanchor 206A is transitioned into a partially deployed shape in which endoanchor 206A pierces stent graft 210 and blood vessel wall 212. Endoanchor 206A includes proximal end 214 and distal end 216. In the partially deployed state, endoanchor 206A includes curved portion 218 extending between proximal end 214 and distal end 216. Curved portion 218 curls into straight portion 220, which terminates at distal end 216. As shown in FIG. 2C, straight portion 220 is aligned with stent graft 210 and blood vessel wall 212. Endoanchor 206A transitions from a straight configuration in the first partially deployed state into the second partially deployed state as endoanchor 206A pierces stent graft 210 and blood vessel wall 212. While extending endoanchor 206A exerts sufficient force to pierce blood vessel wall 212, blood vessel wall 212 exerts sufficient opposite force such that endoanchor 206A bends to form curved portion 218 as endoanchor 206A is advanced. Distal end 216 of endoanchor 206A may have a sharpened point to aid in piercing stent graft 210 and blood vessel wall 212. In one or more embodiments, the pre-set curved shape of endoanchor 206A aids in the bending of endoanchor 206A as it passes through stent graft 210 and blood vessel wall 212. As shown in FIG. 2C, curved portion 218 bridges first location 222 and second location 224 where endoanchor 206A pierces stent graft 210 and blood vessel wall 212. Distal end 216 may therefore pierce the stent graft 210 and the blood vessel wall 212 twice, once on initial deployment (first location 222) and again upon curving back towards the applier catheter 202 (second location 224) to re-enter the lumen of the aorta.

FIG. 2D depicts a side view of endoanchor 206A in a deployed state. As shown in FIG. 2D, endoanchor 206A is transitioned into a fully deployed shape in which endoanchor 206A is expelled from the applier catheter 202 and pierces stent graft 210 and blood vessel wall 212 between first location 222 and second location 224, proximal end 214 and distal end 216 contact stent graft 210 at third location 226 and fourth location 228, between first location 222 and second location 224. In one or more embodiments, proximal end 214 and distal end 216 do not pierce stent graft 210 such that stent graft 210 protects blood vessel wall 212 from edges (e.g., sharp edge(s)) of proximal end 214 and/or distal end 216. In one or more embodiments, endoanchor 206A is symmetrical a plane perpendicular stent graft 210 and/or blood vessel wall 212. The fully deployed shape may be complimentary or congruent the pre-set, curved shape of endoanchor 206A. The pre-curved shape is configured to permit a secure anchor to be left in place in stent graft 210 and blood vessel wall 212. Once endoanchor 206A is fully deployed, applier catheter 202 may be moved to a second deployment location for endoanchor 206B, and then the steps set forth above may be repeated. This sequence may be repeated for endoanchor 206C and endoanchor 206D. The capability to implant multiple endoanchors without removing the endoanchor delivery system may reduce procedure time.

In one or more embodiments, the endoanchors (e.g., endoanchors 206A through 206D) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy to assist with orientation. For example, a letter “E” or letter “C” may be used, as this allows the physician to know whether the marker is facing forwards or backwards.

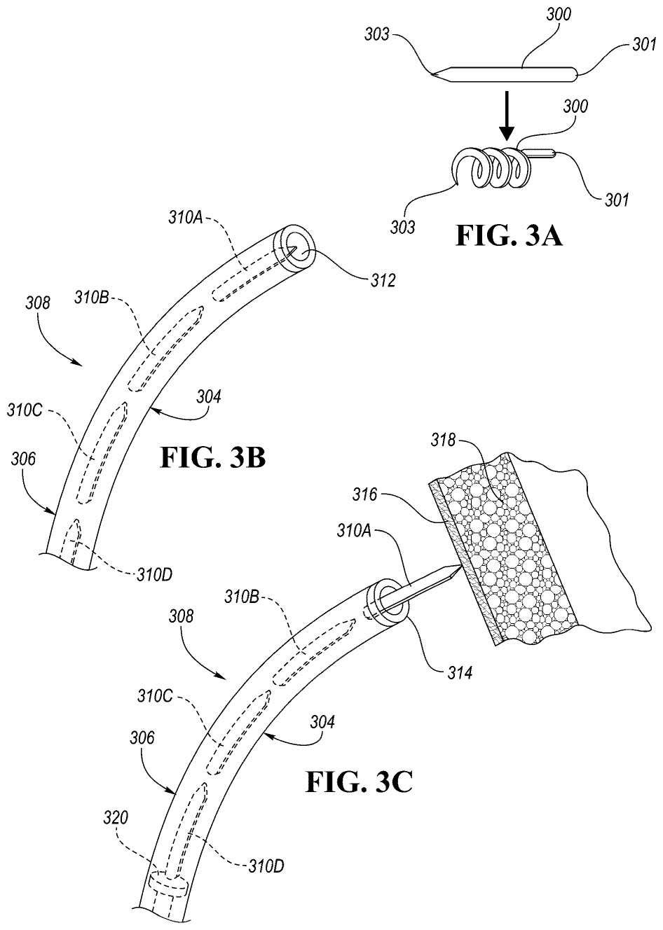

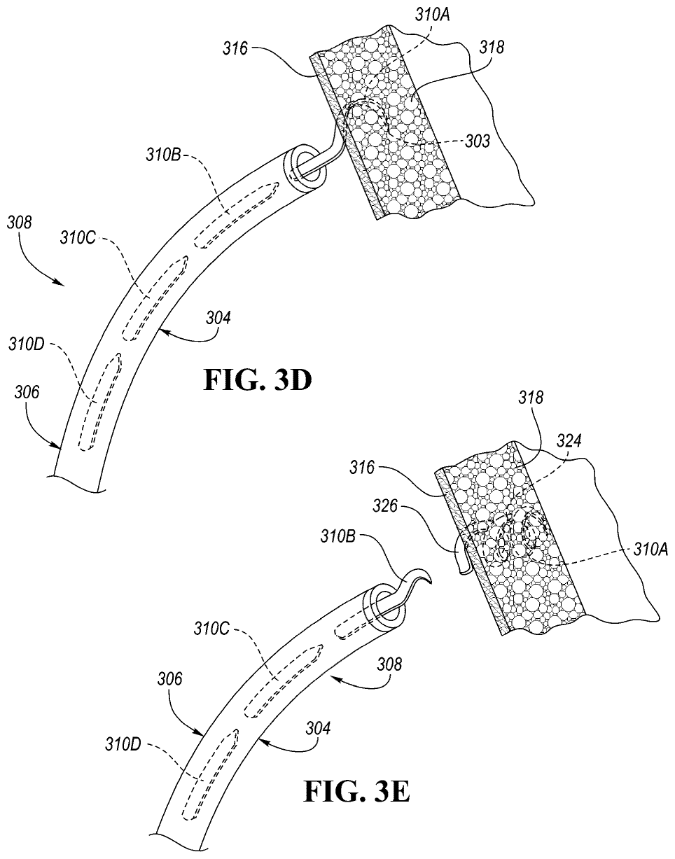

FIG. 3A are perspective, side views of endoanchor 300 in a loaded state and a deployed state. As shown in FIG. 3A, endoanchor 300 has a first surface and an opposing second surface. Endoanchor 300 further includes a peripheral side and thickness, both extending between the first surface and the second surface. As shown in FIG. 3A, endoanchor 300 has a curved proximal end 301 and a pointed distal end 303. The endoanchor may be formed of a shape memory material. The shape memory material may be a shape memory alloy such as Nitinol. The shape memory material may be used to pre-set the deployed state shape of endoanchor 300. The pre-set shape transitions to a different loading shape as described herein. The endoanchors may be shape set in the pre-set shape so that they grip into a blood vessel wall upon deployment of the endoanchors.

Endoanchor 300 in a loaded state has a straight, flat shape (e.g., a straight shape characteristic and a flat shape characteristic) in a constrained configuration. The straight shape characteristic may refer to endoanchor 300 in the loaded state substantially following a longitudinal axis of applier catheter 306. Substantially following may refer to following the longitudinal axis with no or slight deviations from the longitudinal axis. The percentage of slight deviations from the longitudinal axis may be any of the following percentages or in a range of any two of the following percentages: 0.1%, 0.5%, 1%, 2%, 3%, 4%, and 5% (with a 100% deviation signifying that the deviation is normal the longitudinal axis). The flat shape characteristic may refer to the opposing surface(s) having significant flat area regions in a perfectly flat plane. Significant may be any of the following percentages or in a range of any two of the following percentages: 50%, 60%, 70%, 80%, 90%, and 100%.

Endoanchor 300 in a deployed state has a helical, flat shape (e.g., a helical shape characteristic and a flat shape characteristic). The helical, flat shape is configured to pierce and extend through a stent graft and to extend within a blood vessel wall to anchor the stent graft to the blood vessel wall. The helical shape characteristic may include regular space curves with tangent lines at a constant angle to a fixed axis. Regular may refer to the space curves with no or slight deviations from the overall shape of the helix. The percentage of slight deviations from the longitudinal axis may be any of the following percentages or in a range of any two of the following percentages: 0.1%, 0.5%, 1%, 2%, 3%, 4%, and 5%. The fixed axis may be the longitudinal axis of endoanchor 300 in a loaded state. The helical shape characteristic may be a circular helix shape having a constant radius, curvature, and/or torsion. The flat shape characteristic may refer to the opposing surface(s) having significant flat area regions in a perfectly flat plane. Significant may be any of the following percentages or in a range of any two of the following percentages: 50%, 60%, 70%, 80%, 90%, and 100%.

FIG. 3B is a perspective view of distal portion 304 of applier catheter 306 of endoanchor delivery system 308. FIG. 3B depicts first endoanchor 310A, second endoanchor 310B, third endoanchor 310C, and fourth endoanchor 310D loaded into distal portion 304 of applier catheter 306 in the loaded state. In one or more embodiments, upon loading into applier catheter 306, the endoanchors transition into a straight, flat shape from the pre-set helical, flat shape to conform with the reduced volume defined by lumen 312. With the straight, flat shape, multiple endoanchors may be stacked within applier catheter 306 along the longitudinal axis of applier catheter 306. As shown in FIG. 3B, first endoanchor 310A, second endoanchor 310B, third endoanchor 310C, and fourth endoanchor 310D may be spaced apart from each other in the loaded state. In other embodiments, a portion of the peripheral side of one or more adjacent endoanchors may contact each other or the one or more adjacent endoanchors may partially overlap each other along the longitudinal axis of applier catheter 306. Applier catheter 306 includes an outer surface and an inner surface. In one or more embodiments, the inner surface of applier catheter 306 includes fixtures (e.g., rails and/or flats) configured to fix the loaded endoanchors in the loaded straight, flat shape while permitting the endoanchors to translate proximally to distally along the longitudinal axis of applier catheter 306.

FIG. 3C depicts a perspective view of distal portion 304 of applier catheter 306 of in a first partially deployed state. As shown in FIG. 3C, endoanchor 310A partially extends beyond distal end 314 of applier catheter 306. In the first partially deployed state, endoanchor 310A may contact stent graft 316, which is deployed against blood vessel wall 318. Plunger 320 may advance endoanchor 310A (as well as endoanchor 310B, endoanchor 310C, and endoanchor 310D) through lumen 312 of applier catheter 306. Plunger 320 may directly or indirectly push the proximal most endoanchor, which in turn, pushes forward the adjacent endoanchor, where the force is eventually transferred to endoanchor 310A. Alternatively or additionally, applier catheter 306 may be rotated to wind endoanchor 310A into the helical shape. This rotation may also be used to situate the subsequent endoanchor (e.g., endoanchor 310B) for deployment.

FIG. 3D depicts a perspective view of endoanchor 310A in a second partially deployed state. As shown in FIG. 3D, endoanchor 310A is transitioned into a partially deployed shape in which endoanchor 310A pierces stent graft 316 and blood vessel wall 318. In the partially deployed state, endoanchor 310A includes curved portion 322 carrying pointed distal end 303. Endoanchor 310A transitions from a straight configuration in the first partially deployed state into the second partially deployed state as endoanchor 310A pierces stent graft 316 and blood vessel wall 318. While extending endoanchor 310A exerts sufficient force to pierce blood vessel wall 318 and endoanchor 310A begins to transition into its pre-set shape upon exiting lumen 312, blood vessel wall 318 exerts sufficient opposing force that may help endoanchor 310A bend to form curved portion 322 as endoanchor 310A is advanced. Pointed distal end 303 is configured to aid in piercing stent graft 316 and blood vessel wall 318.

FIG. 3E depicts a perspective view of endoanchor 310A in a deployed state. As shown in FIG. 3E, endoanchor 310A is transitioned into a fully deployed shape of a helical, flat shape in which endoanchor 310A is expelled from the applier catheter 306 and pierces stent graft 316 and blood vessel wall 318. As shown in FIG. 3E, the helical, flat shape of endoanchor 310A includes leading surface 324 facing toward stent graft 316 and blood vessel wall 318 and trailing surface 326 facing away from stent graft 316 and blood vessel wall 318. In one or more embodiments, the leading and/or trailing surfaces have flat shape characteristic(s) to reduce drag as endoanchor 310A extends through stent graft 316 and within blood vessel wall. Once endoanchor 310A is fully deployed, applier catheter 306 may be moved to a second deployment location for endoanchor 310B, and then the steps set forth above may be repeated. The sequence may be repeated for endoanchor 310C and endoanchor 310D. The capability to implant multiple endoanchors without removing the endoanchor delivery system may reduce procedure time. The endoanchors may also be used with existing applier catheters and the designs and/or profile sizes thereof.

By using a straight shape characteristic in the loaded state and a helical shape characteristic in the deployed state, the outer diameter of the applier catheter may be reduced while maintaining a helical shape characteristic for the deployed endoanchors. Since the lumen of the applier catheter is relatively smaller and occupied with endoanchors transitioning from a straight configuration to a helical configuration, this arrangement may act as a barrier to blood flowing into the lumen. The pre-set helical shape of the endoanchor may also help the endoanchor thread itself into the vessel wall as it transitions from the straight configuration to the helical configuration.

In one or more embodiments, the endoanchors (e.g., endoanchors 310A through 310D) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy to assist with orientation. For example, a letter “E” or letter “C” may be used, as this allows the physician to know whether the marker is facing forwards or backwards.

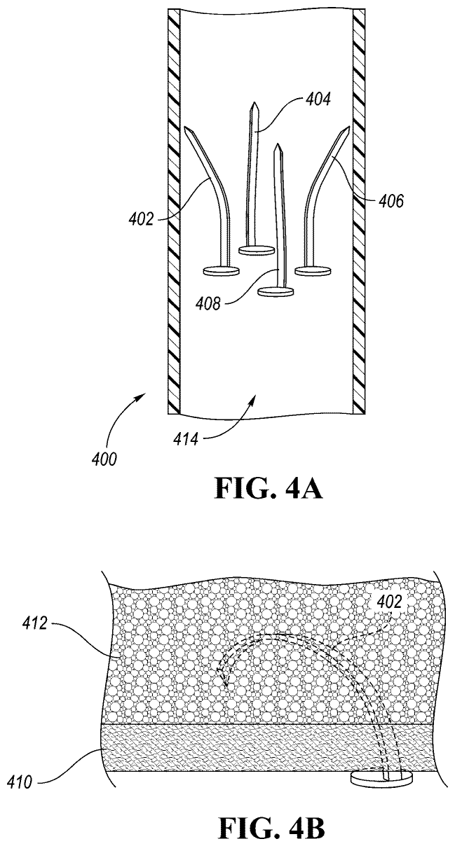

FIG. 4A depicts a cross section view of applier catheter 400 and a side view of first endoanchor 402, second endoanchor 404, third endoanchor 406, and forth endoanchor 408 loaded into applier catheter 400 in a loaded state. FIG. 4B is a side view of endoanchor 402 in a deployed state within stent graft 410 and blood vessel wall 412.

In one or more embodiments, the endoanchors shown in FIG. 4A have a pre-set hooked shape with a curved, extending body and a head having a cross sectional area greater than the cross-sectional area of the curved, extending body. The pre-set hooked shape may change into a generally or entirely constrained, straight shape when loaded into lumen 414 formed by applier catheter 400. The inner surface of applier catheter 400 may include fixtures (e.g., rails and/or flats) to individually and releasable secure the endoanchors within lumen 414. Upon being released, the endoanchors take on their pre-set hooked shape (e.g., the shape of endoanchor 402 shown in FIG. 4B). The endoanchors may be located in a same or substantially same axial position within the applier catheter 400, as shown in FIG. 4A. In this embodiment, a pusher (e.g., as described above) may be used to push all endoanchors out at once to deliver multiple endoanchors in a single step. Alternatively, individual endoanchors may be advanced separately to serially deliver the endoanchors at multiple locations. In another embodiment, the endoanchors may be stacked axially, similar to above, and advanced individually by a plunger.

As shown in FIG. 4B, endoanchor 402 is expelled from the applier catheter 400 and the body of endoanchor 402 extends through stent graft 410 and within blood vessel wall 412 and the head is configured to resist further movement of endoanchor 402 through stent graft 410 and within blood vessel wall 412. The curved, distal portion of endoanchor 402 is configured to anchor stent graft 410 to blood vessel wall 412.

In one or more embodiments, the endoanchors (e.g., endoanchors 402 through 408) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy in order to assist with orientation. For example, a letter “E” or letter “C” may be used, as this allows the physician to know whether the marker is facing forwards or backwards.

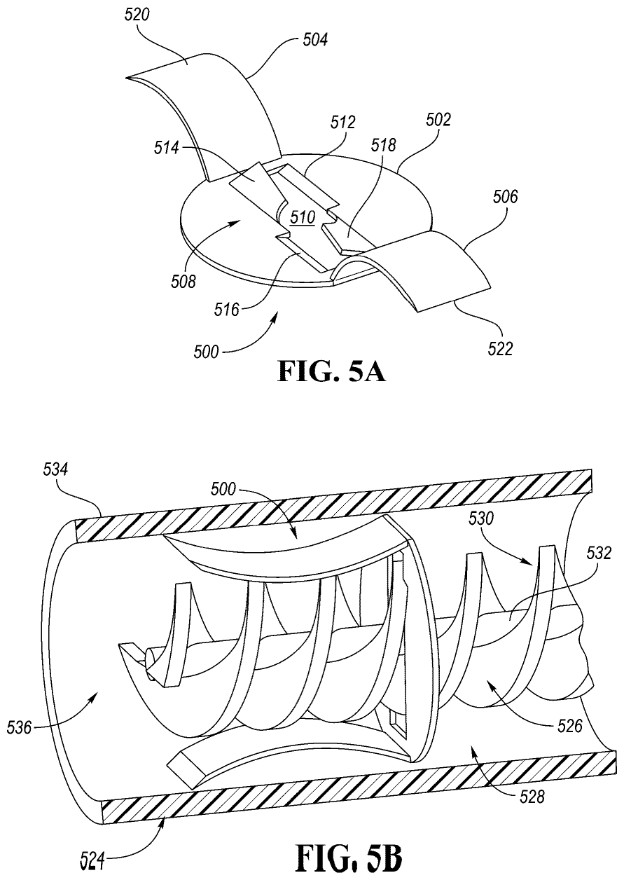

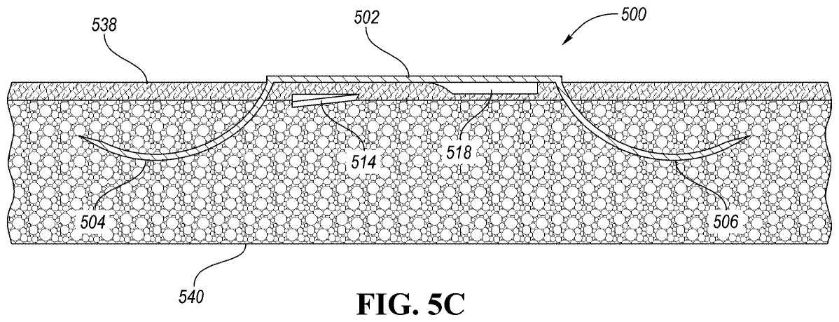

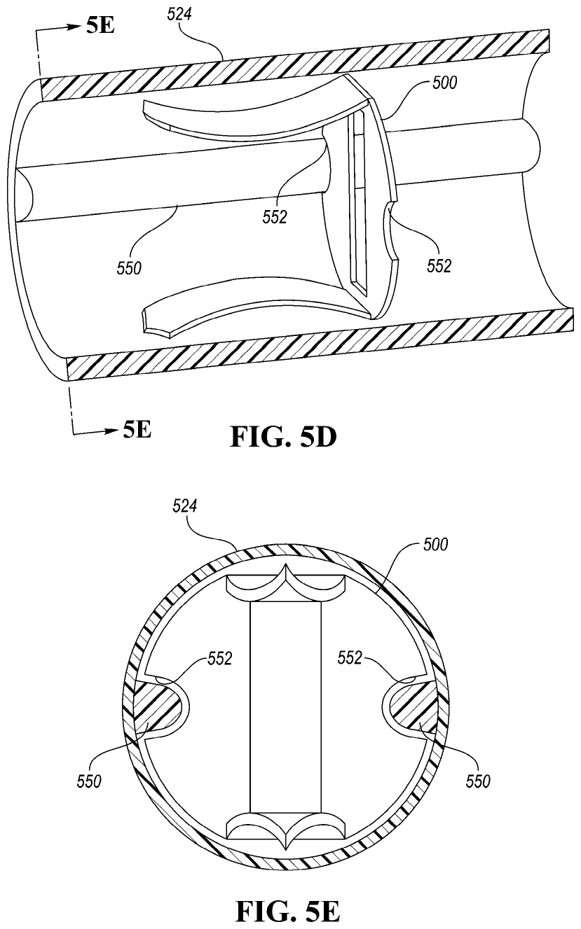

FIG. 5A is a perspective view of endoanchor 500 having base 502 and first leg 504 and second leg 506 extending from base 502. Base 502 includes nut feature 508 configured to permit endoanchor 500 to travel on a screw gear as described herein. Nut feature 508 defines opening 510 to receive the screw gear. Nut feature also includes first ramp 512, first angled tab 514, second ramp 516, and second angled tab 518, collectively or individually configured to interact with a screw gear or other inner shaft for relative movement along the screw gear or other inner shaft. First ramp 512 and second ramp 516 are diagonal each other across opening 510. First angled tab 514 and second angled tab 518 are diagonal each other across opening 510.

First leg 504 and second leg 506 outwardly curve from a surface of base 502. As shown in FIG. 5A, first leg 504 and second leg 506 are in a pre-set shape. First leg 504 and second leg 506 are flexible such that they can flex upward in a constrained configuration within a lumen of an applier catheter including a screw gear or other inner shaft. First leg distal end 520 and second leg distal end 522 may have sharp edges configured to pierce a stent graft and a blood vessel wall. In one or more embodiments, the first and second leg distal ends may be squared, pointed, rounded, or any combination thereof to enhance the ability of first leg 504 and second leg 506 to insert into travel within applier catheter 524 and insert into the blood vessel wall. Although endoanchor 500 is shown with two legs, the endoanchor may include additional legs (e.g., 3, 4, 5, 6, 7, 8, 9, 10 legs or a range of any two of those values). The legs of endoanchor may be formed of a shape memory material. The shape memory material may be a shape memory alloy such as Nitinol.

FIG. 5B is a perspective view of endoanchor 500 in a loaded in applier catheter 524 and mounted on distal portion 526 of inner shaft 528 of an endoanchor delivery system. As shown in FIG. 5B, distal portion 526 of inner shaft 528 is formed by welding flexible threaded rod 530 (e.g., flat wire spring) over guidewire 532 to form a helical thread with a center shaft. The flat wire spring may be formed of stainless steel or other relative hard material configured to permit the outwardly deflecting endoanchors to slide along lumen 536 of applier catheter 524. In one or more embodiments, the guidewire has a relative stiffness that permits some flexing so that applier catheter 524 can transition to a deployment position.

Distal portion 526 may have a length to load multiple staples (e.g., 2, 3, 4, 5, 6, 7, 8, 9, or 10 staples or a range of any two of those values). In one or more embodiments, distal portion 526 only has a length necessary accommodate a certain number of staples. Proximal portion (not shown) of inner shaft 528 extends from distal portion 526. Proximal portion may be welded to distal portion 526 and extend into a handle of endoanchor delivery system. Proximal portion may be a flexible drive shaft configured to transfer torque over a relatively long distance of the delivery system. Proximal portion may have a larger diameter than the diameter of flexible threaded rod 530, and proximal portion may extend to the handle of the endoanchor delivery system.

Flexible threaded rod 530 receives endoanchor 500 through opening 510. Flexible threaded rod 530 is configured to advance endoanchor 500 so it releases from distal end 534 of applier catheter 524 to deploy endoanchor 500. Flexible threaded rod 530 may be configured to hold inwardly deflecting endoanchors open until deployment, while in embodiments with outwardly deflecting endoanchors (e.g., FIG. 5A) they may be constrained by the applier catheter 524. Multiple endoanchors may be loaded in series inside lumen 536 of applier catheter 524. The multiple endoanchors may be loaded inside lumen 536 of applier catheter 524 at the manufacturing site for the endoanchor delivery system. In the operating room, the clinician inserts applier catheter 524 into the patient and deploy up to the amount of endoanchors loaded into the endoanchor delivery system. Applier catheter 524 is configured to be manipulated to be aimed at the endoanchor attachment site (e.g., by a deflectable guide catheter).

FIG. 5C is a cross section view of endoanchor 500 in a deployed state anchoring stent graft 538 to blood vessel wall 540 where endoanchor 500 is expelled from the applier catheter 524. First leg 504 and second leg 506 are configured to bend outward when released from applier catheter 524 and through stent graft 538 and into blood vessel wall 540. During the deployment of endoanchor 500, base 502 is pulled toward stent graft 538 such that first angled tab 514 and second angled tab 518 are pulled into, and may penetrate, the graft material of stent graft 538 as first leg 504 and second leg 506 flex outward into blood vessel wall 540. Opening 510 of endoanchor 500 rides over the helical track of threaded rod 530. Once released from threaded rod 530, base 502 of endoanchor 500 is a stop configured to resist further movement once first leg 504 and second leg 506 are pulled into blood vessel wall 540.

In one or more embodiments, the endoanchor (e.g., endoanchor 500) may include a radiopaque marking material (e.g., gold, platinum, iridium, etc.) configured to be visualized under fluoroscopy. The use of a radiopaque marking material is configured to aid the clinician in deploying each endoanchor in an appropriate location. In at least one embodiment, the endoanchor(s) may have one or more radiopaque markers at their tip or leading edge (e.g., tip of a helical anchor) to assist with visualization of the endoanchor as it is being positioned and/or inserted. In another embodiment, the endoanchor(s) may alternatively or additionally have one or more radiopaque markers on their base or the trailing edge to assist with visualization of endoanchor during and/or after deployment. The radiopaque markers may have a shape that is asymmetrical on at least one axis in a plane visible under fluoroscopy to assist with orientation. For example, a letter “E” or letter “C” may be used, as this allows the physician to know whether the marker is facing forwards or backwards.

FIG. 5D is a cross section view of applier catheter 548 including rails 550 matching notches 552 in endoanchor 546. FIG. 5E is a cross section view of applier catheter 548 taken along line 5E-5E of FIG. 5D and showing rails 550 matching notches 552 of endoanchor 546. Rails 550 extend radially inward the inner surface of applier catheter 548. Rails 550 may be formed integral to applier catheter 548 or may be separate pieces secured to the inner surface of applier catheter 548. In one or more embodiments, rails 550 are configured to resist the endoanchors from rotating with the threaded shaft (e.g., flexible threaded rod 530) when the threaded shaft is rotated. In one or more embodiments, the rails are configured to allow the endoanchors to advance in an axial direction without rotating so that legs 520 and 522 pierce the graft material and the vessel wall in an axial manner and do not rotate or thread themselves into the vessel wall, which may be too traumatic to the vessel wall given the shape of the legs.

In one or more embodiments, the profile of rails 550 matches the profile of notches 552 to resist the endoanchor from rotating within applier catheter 524 when the threaded shaft is rotated and/or to provide axial movement of the endoanchors within applier catheter 548. As shown in FIGS. 5D and 5E, rails 550 and notches 552 have semi-circular profiles. While two rail/notch combinations are shown in FIGS. 5D and 5E, in other embodiments, more than two combinations (e.g., 3, 4, or 5) may be used.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. An endoanchor delivery system comprising:
a handle system;
an applier catheter extending from the handle system and defining a lumen therein, the applier catheter having a proximal portion and a distal portion; and
a series of endoanchors loaded into the lumen of the applier catheter at the distal portion thereof and disposed along a longitudinal axis of the applier catheter in a loaded state, the series of endoanchors include a first endoanchor and a second endoanchor, the first endoanchor is located distalmost the applier catheter in the loaded state, the second endoanchor is located inward the first endoanchor along the longitudinal axis of the applier catheter, the first endoanchor has a straight, flat shape in the loaded state, the first endoanchor has a helical, flat shape in a deployed state in which the first endoanchor anchors an implant to a blood vessel wall.

2. The endoanchor delivery system of claim 1, wherein the first endoanchor includes a first surface and a second surface opposing the first surface, the first surface and/or the second surface having a flat shape characteristic.

3. The endoanchor delivery system of claim 1, wherein the first endoanchor in the deployed state has a first endoanchor deployed circumference, the applier catheter has an outer surface and an inner surface, the inner surface has an inner surface circumference less than the first endoanchor deployed circumference.

4. The endoanchor delivery system of claim 1, wherein the helical, flat shape is a pre-set helical, flat shape.

5. The endoanchor delivery system of claim 4, wherein the first endoanchor is formed of a shape memory material to set the pre-set helical, flat shape.

6. The endoanchor delivery system of claim 1, wherein the straight, flat shape of the first endoanchor in the loaded state includes a straight shape characteristic where the first endoanchor substantially follows the longitudinal axis of the applier catheter with deviations of 5% or less.

7. The endoanchor delivery system of claim 6, wherein the helical, flat shape of the first endoanchor in the deployed state includes a helical shape characteristic of a circular helix shape having a constant radius, curvature, and/or torsion.

8. The endoanchor delivery system of claim 1 further comprising a plunger configured to apply force to the series of endoanchors along the longitudinal axis of the applier catheter to transition the first endoanchor from the loaded state to the deployed state.

9. An endoanchor delivery system comprising:
a handle system;
an applier catheter extending from the handle system and defining a lumen therein, the applier catheter includes an inner shaft having a proximal portion and a distal portion; and
a series of endoanchors loaded into the lumen of the applier catheter and onto the distal portion of the inner shaft thereof in a loaded state, the series of endoanchors includes a first endoanchor and a second endoanchor, the first endoanchor is located distalmost the applier catheter in the loaded state, the second endoanchor is located inward the first endoanchor along a longitudinal axis of the applier catheter, the first endoanchor includes a base and first and second legs extending from the base, the base defines an opening configured to communicate with the inner shaft for axial movement along the longitudinal axis, the first and second legs are in a loaded position in the loaded state, the first and second legs are in a deployed position different than the loaded position in a deployed state in which the first endoanchor anchors an implant to a blood vessel wall.

10. The endoanchor delivery system of claim 9, wherein the first endoanchor includes one or more ramps configured to communicate with the inner shaft for axial movement along the longitudinal axis.

11. The endoanchor delivery system of claim 9, wherein the first endoanchor includes one or more angled tabs configured to pierce the implant when the first endoanchor is in the deployed state.

12. The endoanchor delivery system of claim 9, wherein the base of the first endoanchor is configured to contact the implant when the first endoanchor is in the deployed state.

13. The endoanchor delivery system of claim 9, wherein the first and second legs are outwardly curving relative to the base of the first endoanchor.

14. The endoanchor delivery system of claim 9, wherein the distal portion of the inner shaft includes a helical gear and the proximal portion of the inner shaft has a cylindrical shape and does not include the helical gear.

15. The endoanchor delivery system of claim 14, wherein all the endoanchors in the series of endoanchors are situated on the helical gear of the distal end of the inner shaft in the loaded state.

16. The endoanchor delivery system of claim 14 further comprising one or more fixtures extending from an inner surface of the applier catheter, the one or more fixtures configured to resist a rotation of the first and second endoanchors when the helical gear of the inner shaft is rotated and to permit axial movement of the first and second endoanchors along the longitudinal axis of the applier catheter.

17. A method of deploying a series of endoanchors, the method comprises:
loading a series of endoanchors serially within a lumen defined by a distal portion of an applier catheter, the series of endoanchors including a first endoanchor and a second endoanchor, the first endoanchor is located distalmost the applier catheter in a loaded state, the second endoanchor is located inward the first endoanchor along a longitudinal axis of the applier catheter, the first endoanchor includes a constrained shape and a pre-set shape, the first endoanchor is in the constrained shape in the loaded state; and
sequentially deploying the series of endoanchors including expelling the first endoanchor from the lumen of the applier catheter such that the first endoanchor transitions from the constrained shape into the pre-set shape in which the first endoanchor anchors an implant to a blood vessel wall.

18. The method of claim 17, wherein the first endoanchor includes a proximal end and a distal end, the sequentially deploying step for the first endoanchor includes a first partially deploying step and a second partially deploying step, the first partially deploying step includes contacting the stent graft with the distal end of the first endoanchor, and the second partially deploying step includes piercing the implant and the blood vessel wall with the first endoanchor.

19. The method of claim 17, wherein the first and second endoanchors are spaced apart from each other in the constrained shape along the longitudinal axis of the applier catheter.

20. The method of claim 17, wherein the loading step includes loading the series of endoanchors from a distal opening of the applier catheter.
